(19) United States
(12) Patent Application Publication
(10) Pub. No.: US 2025/0279184 A1
(43) Pub. Date: Sep. 4, 2025

(54) PERSONALITY TRAIT ESTIMATION SYSTEM, PERSONALITY TRAIT ESTIMATION METHOD, AND COMPUTER PROGRAM

(71) Applicant: Hitachi, Ltd., Tokyo (JP)

(72) Inventors: Toshiteru NAKAMURA, Tokyo (JP); Takashi NUMATA, Tokyo (JP); Takuya NAKAMICHI, Tokyo (JP)

(21) Appl. No.: 18/907,626

(22) Filed: Oct. 7, 2024

(30) Foreign Application Priority Data
Feb. 29, 2024 (JP) 2024-030608

(51) Int. Cl. G16H 20/70

(52) U.S. Cl. CPC G16H 20/70

(57) ABSTRACT

A system receives measurement data related to a behavior performed by a subject and based on measurement performed by one or a plurality of sensors from the subject apparatus, and generates related behavioral data of a related behavior that is an entire or a partial behavior excluding instruction of an intension of the subject from the measurement data. The system acquires one or a plurality of behavioral features that are respectively nonverbal features based on related behavioral data with respect to one or each of a plurality of related behaviors, and estimates a psychological characteristic of the subject based on the one or the plurality of behavioral features. The system outputs estimated psychological characteristic data that is data expressing the psychological characteristic that is estimated.

### CROSS-REFERENCE TO RELATED APPLICATION

The present application claims priority from Japanese application JP2024-030608, filed on Feb. 29, 2024, the content of which is hereby incorporated by reference into this application.

### BACKGROUND OF THE INVENTION

### 1. Field of the Invention

The present invention generally relates to a technique for estimating a personality trait.

### 2. Description of the Related Art

As an example of a personality trait, a psychological characteristic (personality) is named. As a technique for estimating a psychological characteristic, for example, a technique disclosed in U.S. Pat. No. 10,957,306 is known. The technology disclosed in U.S. Pat. No. 10,957,306 estimates a psychological characteristic of a user based on text data in addition to utterance data of the user.

As a technique for estimating a psychological state of a person, techniques disclosed in JP 2022-179438 A and JP 2023-061407 A are known. The technique disclosed in JP 2022-179438 A estimates a psychological state of a user based on vital data of the user. The technique disclosed in JP 2023-061407 A estimates a psychological characteristic of a student based on a state where the student is taking a lecture.

### SUMMARY OF THE INVENTION

In the technique disclosed in U.S. Pat. No. 10,957,306, a subject has to answer questions prepared for a purpose different from the psychological characteristic estimation. Further, the subject has to take another behavior such as utterance for the psychological characteristic estimation, it is not always the case that the subject takes such another behavior. Accordingly, it is not always the case that the psychological characteristic is estimated with respect to the subject. Further, there may arise a problem that it takes a lot of time to prepare text data as an input.

In the techniques disclosed in JP 2022-179438 A and JP 2023-061407 A, although a temporary psychological state is estimated. However, these techniques cannot estimate psychological characteristics unique to a person.

A system receives measurement data related to a behavior performed by a subject and based on measurement performed by one or a plurality of sensors from the subject apparatus, and generates related behavioral data of a related behavior that is an entire or a partial behavior excluding instruction of an intention of the subject from the measurement data. The system acquires one or a plurality of behavioral features that are respectively nonverbal features based on related behavioral data with respect to one or each of a plurality of related behaviors, and estimates a psychological characteristic of the subject based on the one or the plurality of behavioral features. The system outputs estimated psychological characteristic data that is data expressing the psychological characteristic that is estimated.

According to the present invention, the psychological characteristic of the subject can be estimated without making the subject perform a behavior dedicated for estimating the psychological characteristics.

### BRIEF DESCRIPTION OF THE DRAWINGS

FIG. 1 illustrates a configurational example of an entire system according to a first embodiment;

FIG. 2 illustrates data and functions in the entire system according to the first embodiment;

FIG. 3 illustrates an example of a flow of processing performed in the first embodiment;

FIG. 4 illustrates an example of a relationship between a subject, details of the subject, data items, and behavioral features;

FIG. 5 illustrates an example of the detection of a person and a landmark within a camera frame;

FIG. 6A illustrates an example of the distribution of behavioral features related to a head;

FIG. 6B illustrates an example of the distribution of behavioral features related to a shoulder;

FIG. 7 illustrates an example of a relationship between harmonicity and a maximum answer time;

FIG. 8 illustrates data and functions in an entire system according to a second embodiment;

FIG. 9 is an explanatory diagram of an example of the determination of outliers; and

FIG. 10 is an example of a practical application of a personality trait estimation system according to the first and second embodiments.

### DESCRIPTION OF THE PREFERRED EMBODIMENTS

In the following description, “interface apparatus” may be one or more interface devices. One or more interface devices may be at least one of the following interface devices.
An I/O interface apparatus that is one or more input/output (I/O) interface devices. An input/output (I/O) interface device is an interface device for at least one of the I/O device and a remote display computer. The I/O interface device for the display computer may be a communication interface device. At least one I/O device may be a user interface device, for example, an input device such as a keyboard and a pointing device, or an output device such as a display device.
A communication interface apparatus that is one or more communication interface devices. One or more communication interface devices may be one or more communication interface devices of the same type (for example, one or more network interface cards (NIC)) or two or more communication interface devices of different types (for example, an NIC and a host bus adapter (HBA)).

In the following description, “memory” is one or more storage devices that are an example of one or more storage devices, and may typically be a main storage device. At least one storage device in the memory may be a volatile storage device or a nonvolatile storage device.

Further, in the following description, “persistent storage apparatus” may be one or more persistent storage devices that are an example of one or more storage devices. Typically, the persistent storage device may be a nonvolatile storage device (for example, an auxiliary storage device). Specifically, the persistent storage device may be, for example, a hard disk drive (HDD), a solid state drive (SSD), a nonvolatile memory express (NVME) drive, or a storage class memory (SCM).

Further, in the description made hereinafter, a “storage apparatus” may be at least a memory out of a memory and a persistent storage apparatus.

In the description made hereinafter, a “processor” may be one or more processor devices. At least one processor device may typically be a microprocessor device such as a central processing unit (CPU). However, the processor device may be a processor device of other type such as a graphics processing unit (GPU). At least one processor device may be a single core or a multi-core. At least one processor device may be a processor core. At least one processor device may be a processor device in a broad sense such as a circuit that is an aggregate of gate arrays in a hardware description language that performs a part of or all processing (for example, a field-programmable gate array (FPGA), a complex programmable logic device (CPLD) or an application specific integrated circuit (ASIC)).

In the description made hereinafter, a function may be described using an expression “yyy unit”. However, the function may be realized by allowing a processor to execute one or more computer programs, may be realized by one or more hardware circuits (for example, FPGA or ASIC), or may be realized by a combination of these hardware circuits. In a case where the function is realized by allowing the processor to execute the program, predetermined processing is suitably performed using the storage apparatus and/or the interface apparatus and hence, the function may be considered as at least a part of the processor. The processing described using a function as a subject may be considered as processing performed by a processor or an apparatus that includes the processor. A program may be installed from a program source. The program source may be, for example, a program distribution computer or a computer-readable storage medium (for example, a non-transitory storage medium). The description of each function is an example. Accordingly, a plurality of functions may be integrated into one function, or one function may be divided into a plurality of functions.

Further, in the description made hereinafter, in a case where elements of the same kind are described without distinguishing them from each other, a common signal is used among reference signals, and in a case where elements of the same kind are described for distinguishing them from each other, the reference signals may be used.

Some embodiments will be described hereinafter. In the embodiment described hereinafter, a psychological characteristic is adopted as an example of the personality trait, and the psychological characteristic is estimated.

### First Embodiment

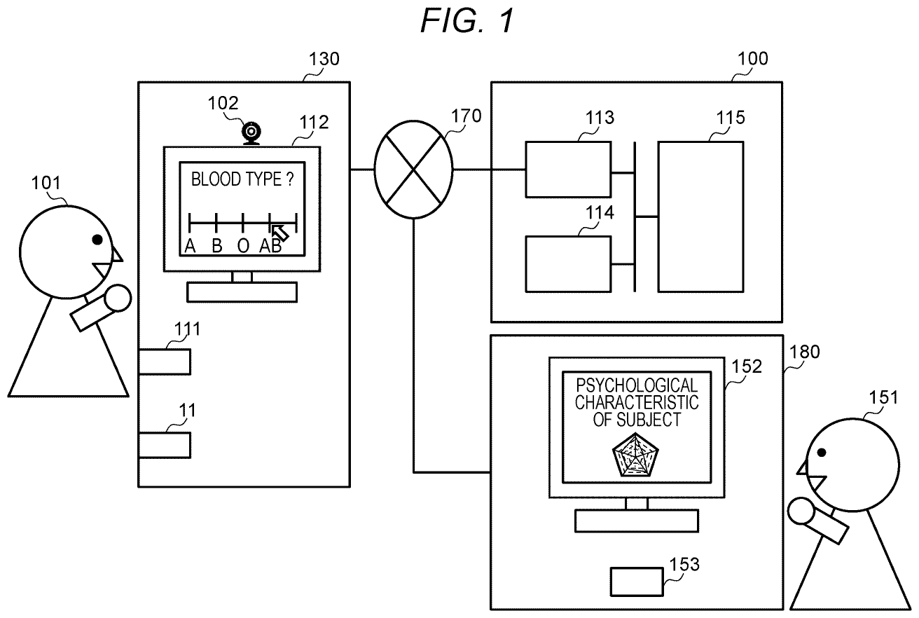

FIG. 1 illustrates a configurational example of an entire system according to a first embodiment.

A personality trait estimation system 100 communicates with a subject apparatus 130 and an administrator apparatus 180 via a communication network 170. The communication network 170 is, for example, the Internet, a wide area network (WAN), or a local area network (LAN).

The subject apparatus 130 is an information processing terminal, for example, a computer such as a personal computer or a smartphone of a subject 101. The subject apparatus 130 includes: one or a plurality of sensors that measure a behavior of the subject 101; and a display device 112. One or more sensors are formed of, for example, a camera 102, an input device 111 (for example, a keyboard and a pointing device), or a microphone 11. In place of or in addition to the input device 111, the display device 112 may be a touch panel. Furthermore, the subject 101 may be, for example, an applicant in an online interview.

The administrator apparatus 180 is an information processing terminal, for example, a computer such as a personal computer or a smartphone of the administrator 151. The administrator apparatus 180 includes an input device 153 and a display device 152. The administrator 151 may be, for example, an interviewer in an online interview. In a case where the online interview is a self-interview, an interviewer is a virtual robot such as an avatar. In this case, the administrator 151 may not be an interviewer, and may be a person who evaluates the subject 101 based on the result of estimation by the personality trait estimation system 100.

The personality trait estimation system 100 includes an interface apparatus 113, a storage apparatus 114, and an arithmetic operation apparatus 115 that is connected to the interface apparatus 113 and the storage apparatus 114.

The interface apparatus 113 communicates with the subject apparatus 130 and the administrator apparatus 180 via the communication network 170. The storage apparatus 114 stores: a computer program executed by the arithmetic operation apparatus 115; and data that is inputted to and outputted from the arithmetic operation apparatus 115. The arithmetic operation apparatus 115 is a processor and executes a computer program.

The arithmetic operation apparatus 115 provides the subject apparatus 130 with the provided information that is information including induction information for inducing the subject 101 to take a behavior for achieving a purpose that differs from the psychological characteristic estimation. The induction information may include a plurality of (or one) questions. Specifically, the induction information may include, for example, a plurality of (or one) questions that are provided by a voice and/or a text by executing an artificial intelligence (AI) or other program that works as an interviewer, and a content of a virtual robot such as an avatar that provides these questions. The content may include a text on which questions are described or other types of information (for example, graphics). The “questions” provided in the present embodiment may be general questions in an interview, and may not include questions prepared for the psychological characteristic estimation.

The arithmetic operation apparatus 115 receives measurement data that is related to the behaviors performed by the subject 101 who is induced by induction information of the provided information and is obtained based on the measurement performed by one or a plurality of sensors, from one or a plurality of sensors via the interface apparatus 113.

The arithmetic operation apparatus 115 specifies the subject intention data and the related behavioral data based on the measurement data. The “behavior induced by the induction information” includes the designation of the intention of the subject, and related behaviors that are all or some of behaviors excluding the designation of the intention of the subject. In this embodiment, the designation of the intention of the subject means that the subject answers a question (for example, by type inputting or voice inputting of the answer to the question).

However, the designation of the intention of the subject may take various forms corresponding to information to be provided. In the present embodiment, the related behaviors are all or some behaviors ranging from the providing of the question to the subject to the answering to the question by the subject. However, the related behaviors may also take various forms corresponding to the designation of the intention of the subject that depends on the provided information. The subject intention data is data expressing the designated subject intention. The related behavioral data is data expressing related behaviors.

The arithmetic operation apparatus 115 calculates one or a plurality of behavioral features based on the related behavioral data for each of one or a plurality of related behaviors, and estimates the psychological characteristic of the subject 101 based on one or the plurality of behavioral features. Specifically, for example, each time a question is displayed, the subject 101 answers the displayed question and hence, there is a related behavior for each set of question and answer. The plurality of related behaviors each have the related behavioral data. The arithmetic operation apparatus 115 calculates one or a plurality of behavioral features based on the related behavioral data of the plurality of related behaviors, and estimates the psychological characteristic of the subject 101 based on one or the plurality of behavioral features.

The arithmetic operation apparatus 115 outputs estimated psychological characteristic data that is data expressing the estimated psychological characteristic. For example, the arithmetic operation apparatus 115 transmits the estimated psychological characteristic data to the administrator apparatus 180, and the administrator apparatus 180 displays the psychological characteristic that the estimated psychological characteristic data expresses on the display device 152. Accordingly, the administrator 151 knows the estimated psychological characteristic of the subject 101.

Each of one or more behavioral features used for estimating the psychological characteristics of the subject 101 may be information where at least a part of the provided information is adjusted and/or a behavioral feature enhanced in accordance with an elapsed of time with respect to such a behavioral feature (an example of the enhanced behavior amount described later in detail).

According to this embodiment, it is possible to estimate the psychological characteristics of the subject 101 without making the subject 101 perform behaviors different from the behaviors induced by inducing information (for example, the answers to the questions) that is prepared and is provided for a purpose different from the psychological characteristics estimation, that is, without making the subject 101 perform behaviors dedicated for estimating the psychological characteristics. When one or more behavioral features used for estimating the psychological characteristics of the subject 101 are enhanced, improvement in the estimation accuracy of the psychological characteristics is expected.

The personality trait estimation system 100 may be a physical computer system (one or more physical computers) illustrated in FIG. 1. However, the personality trait estimation system 100 may be a logical computer system (for example, a cloud computing service) based on a physical computer system instead of the physical computer system illustrated in FIG. 1.

Further, the “psychological characteristic” may include one or more psychological characteristic components, and such one or more psychological characteristic components may include at least one of temperament, character, personality, belief, sense of value, mood, and emotion. Furthermore, the arithmetic operation apparatus 115 may perform the estimation of the psychological characteristics based on a part of the subject intention data. Furthermore, the arithmetic operation apparatus 115 may estimate, in addition to the estimated psychological characteristic data, the personality trait (for example, other than the psychological characteristics, at least one of a name, a gender, a date of birth, an age, a motivation, a desired job category, a background, a grade, and a holding skill being included) of the subject 101 based on the subject intention data (for example, answer data including name, gender, and the like).

The “related behavior” may be a behavior leading to an answer to a question (an example of designation of a subject intention). For example, even if the answer is the same, the behavior leading to the answer is influenced by the psychological characteristics of the subject 101. The related behavioral data that expresses such a behavior is used for the estimation of the psychological characteristic. Accordingly, it is expected that the psychological characteristic can be accurately estimated even if there is no question or subject behavior dedicated to the psychological characteristic estimation.

The present embodiment will be described in detail hereinafter.

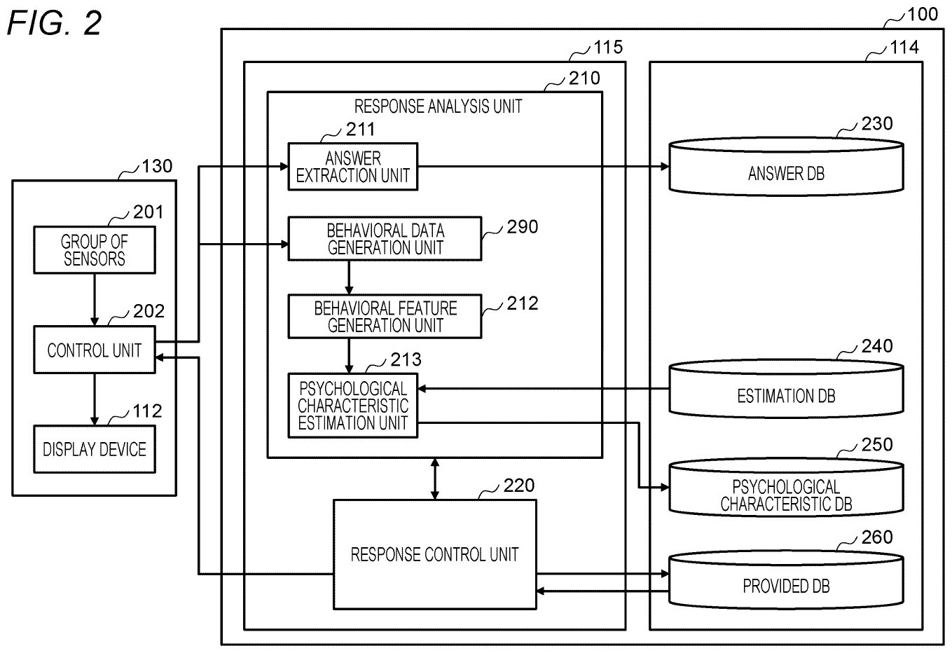

FIG. 2 illustrates data and functions in the entire system according to the first embodiment. In the present embodiment, “DB” is an abbreviation for database. Data may not be structural data such as a database.

As described above, the subject apparatus 130 includes a group of sensors 201 (one or a plurality of sensors) and the display device 112. The subject apparatus 130 also includes a control unit 202. The control unit 202 is implemented by allowing an arithmetic operation apparatus (not illustrated in the drawing) that is a processor of the subject apparatus 130 to execute a program (for example, an application program).

The control unit 202 transmits measurement data obtained based on the measurement performed by the respective sensors in the group of sensors 201 to the personality trait estimation system 100. Furthermore, the control unit 202 outputs the provided information transmitted from the personality trait estimation system 100 to the display device 112 and/or another output device (for example, a speaker).

The storage apparatus 114 stores an answer DB230, an estimation DB240, a psychological characteristic DB250, and a provided information DB260. The answer DB230 is a DB that stores answer data (data expressing answers to questions). The estimation DB240 is a DB that stores data used for estimating psychological characteristics (for example, a model such as one or a plurality of regression equations). The psychological characteristic DB250 is a DB that stores estimated psychological characteristic data (data expressing the estimated psychological characteristic). The provided information DB260 is a DB that stores the provided information (for example, the content itself such as questions) and information related to the information (for example, metadata such as luminance of content).

By allowing the arithmetic operation apparatus 115 to execute a computer program, a response analysis unit 210 that analyzes a response from the subject 101 and a response control unit 220 that controls the response to the subject 101 are implemented. The response analysis unit 210 includes: an answer extracting unit 211 that extracts answers; a behavioral data generation unit 290 that generates related behavioral data; a behavioral feature generation unit 212 that generates behavioral features; and a psychological characteristic estimation unit 213 that estimates psychological characteristics.

Hereinafter, an example of functions realized by the arithmetic operation apparatus 115 and an example of processing performed in the present embodiment will be described.

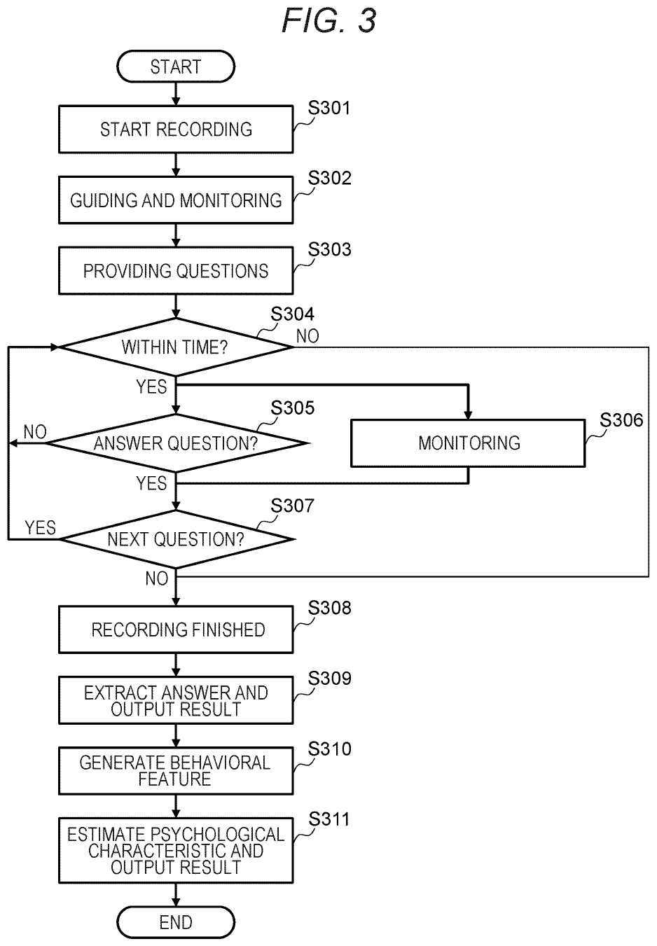

FIG. 3 illustrates an example of a flow of processing performed in the first embodiment.

The response analysis unit 210 receives data of a moving image imaged by the camera 102 in the group of sensors 201 from the control unit 202 of the subject apparatus 130. The response analysis unit 210 starts recording of the moving image (S301). The recorded data (moving image data) may be at least a part of the measurement data, and is stored in the storage apparatus 114 by the response analysis unit 210. The response control unit 220 may transmit a notification of starting of recording to the control unit 202 of the subject apparatus 130, and the control unit 202 may output the notification through the display device 112 or other output devices.

The response control unit 220 offers a guide the subjects 101, and the response analysis unit 210 monitors the subjects 101 who have received the guide (S302). Specifically, the induction information that the response control unit 220 transmits to the subject apparatus 130 includes information indicating a guide to the subject 101. The guide may be a guide that contributes to the enhancement of a behavioral feature. For example, when the response analysis unit 210 determines, from the recorded data, that the position of the subject 101 is not appropriate with respect to an angle of view of the camera 102 (or regardless of whether the position is appropriate or not), the response control unit 220 may transmit, to the subject apparatus 130, the induction information that includes information indicating induction for superimposing the position of the subject 101 on the appropriate position with respect to the angle of view of the camera 102. Instead of or in addition to the guide at the position with respect to the angle of view of the camera 102, the guide may include another guide that contributes to the enhancement of a behavioral feature, for example, a guide for increasing the probability of accurately detecting the utterance of the subject 101 (for example, a guide for adjusting of the setting of a microphone, or a guide for adjusting a distance between the microphone and the subject 101.).

In monitoring, the behavioral data generation unit 290 of the response analysis unit 210 specifies a behavior of the subject 101 with respect to the inducing information that includes the information expressing such a guide (one example of the related behavior) from the recorded data, and generates the related behavioral data expressing the specified behavior. The behavioral feature generation unit 212 generates a behavioral feature based on the related behavioral data. The generated behavioral feature may be stored in the storage apparatus 114.

The number of behavioral features is not limited to one, and psychological characteristics can be estimated by integrating various behavioral features. For example, the related behavioral data specified by the behavioral feature generation unit 212 based on the recorded data may be data expressing at least one consisting of a head motion (a motion of a head of the subject 101); a facial expression (facial expression of the subject 101); an eyeball movement (an eyeball movement of the subject 101); a posture (a posture of the subject 101); a body motion (a body motion of the subject 101); a sound (a sound of utterance of the subject 101); a vital (a vital of the subject 101), a time (a time that the subject 101 requires for a response); and an apparatus operation (an operation performed by the subject 101 on the subject apparatus 130). The behavioral feature generation unit 212 generates a behavioral feature based on such related behavioral data. With such a configuration, the psychological characteristics of the subject 101 can be estimated from at least one viewpoint out of various viewpoints.

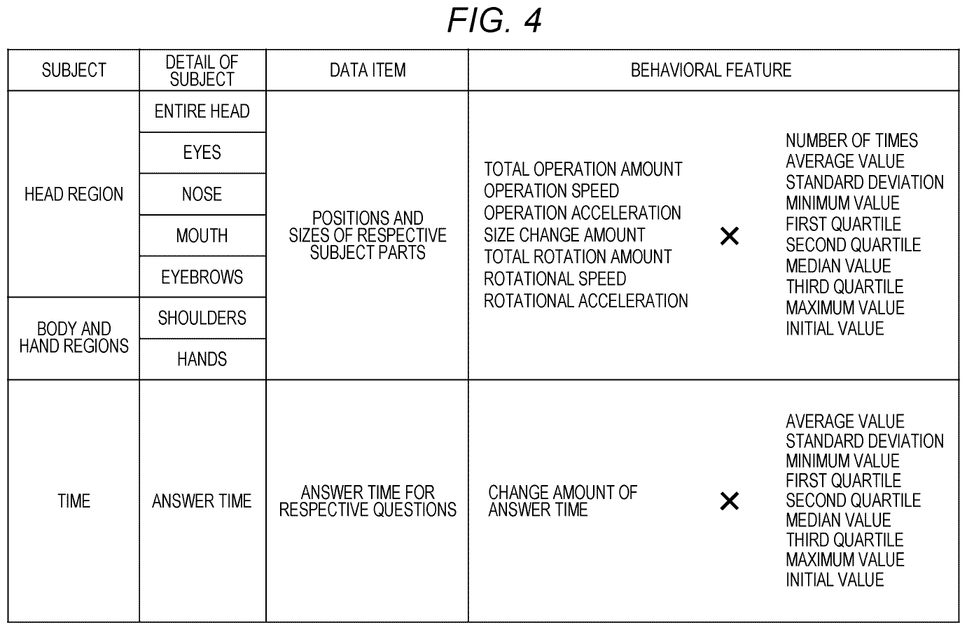

FIG. 4 illustrates an example of a relationship between an object, details of object, data items, and behavioral features. The “object” is an object related to the related behavioral data, and is, for example, a region of a body, or a time related to the related behavior. The “object detail” is a detail of the “object”, and expresses, for example, the movement of which part of the body motion is extracted as a related behavior or what kind of time is the time in which the related behavior is involved. The “data items” are data items of related behavioral data, and are, for example, positions and sizes of part of the body or answer times to the respective questions. In the “behavioral features”, types of related behavior values and types of values that are acquired based on the related behavior values as the behavioral features are defined. The “related behavior values” are values specified from the related behavioral data (values expressing the related behaviors). According to the example illustrated in FIG. 4, as the related behavior values, “a total operation amount”, “an operation speed”, “an operation acceleration”, “a size change amount”, “a total rotation amount”, “a rotational speed”, “a rotation acceleration”, “answer time”, and “a change amount (of the answer time)” are listed. As the behavioral feature, “the number of times”, “an average value”, “a standard deviation”, “a minimum value”, “a first quartile”, “a second quartile”, “a median value”, “a third quartile”, “a maximum value”, and “an initial value” are named Specifically, for example, as a related behavior value that is obtained from related behavioral data specified from recorded data, an operational speed related to a head motion is named. A behavioral feature such as a maximum value or a minimum value of a speed of the head of the subject 101 is obtained from the time series of such an operation speed. The behavioral feature generation unit 212 may generate a behavioral feature by a predetermined generation model (for example, a regression equation or other model) using one or a plurality of related behavior values. The generation model for generating the behavioral feature may differ for each behavioral feature. Further, the generation model for each behavioral feature may be stored in the storage apparatus 114 (for example, the estimation DB240) and specified from the storage apparatus 114.

After the inducing and the monitoring are performed (after S302), the response control unit 220 specifies one or more questions provided from the provided DB260, and provides the provided information that includes information indicating one or a plurality of specified questions to the subject apparatus 130 (S303). The questions may be provided all together or sequentially (a next question being made each time the answering is made).

The response analysis unit 210 may set a time limit for answering one plural questions that are provided, and may notify the subject 101 of the time limit. For example, the time limit of the answer is not necessarily required. However, in a case where the time limit is associated with one or a plurality of questions that are provided in the provided DB260, the response analysis unit 210 may set the time limit.

For example, the response analysis unit 210 determines whether or not the answering is within the time limit (S304). If there is an answer within the time limit (S304: YES, S305: YES), the answer extracting unit 211 of the response analysis unit 210 extracts the answer from the measurement data (data including data expressing the answer inputted via the input device 111) from the subject apparatus 130, and stores data expressing the extracted answer in the storage apparatus 114. In parallel to the above-mentioned processing, the behavioral data generation unit 290 generates related behavioral data from the measurement data, and stores the generated related behavioral data in the storage apparatus 114 (S306).

If there is a next question (S307: YES), the processing returns to S304. In a case where the answering to all questions has been completed and there is no next question (S307: NO) or in a case where the time limit has come (S304: NO), the response analysis unit 210 finishes the recording (S308). The response control unit 220 may transmit a notification of finishing of recording to the control unit 202 of the subject apparatus 130, and the control unit 202 may output the notification through the display device 112 or another output device.

The answer extracting unit 211 acquires the answer data stored in the storage apparatus 114, and outputs (stores) the acquired answer data to the answer DB230 (S309). The answer extracting unit 211 may output the acquired answer data to the administrator apparatus 180.

The behavioral feature generation unit 212 calculates a related behavior value for each related behavior from the related behavioral data for each of related behaviors accumulated in the storage apparatus 114, and generates a plurality of (or one) behavioral features using the plurality of (or one) calculated related behavior values (S310).

The psychological characteristic estimation unit 213 estimates the psychological characteristic of the subject 101 using the model expressed by the estimation DB240 based on a plurality (or one) of the generated behavioral features, and outputs (stores) the estimated psychological characteristic data to the psychological characteristic DB250 (S311). The psychological characteristic estimation unit 213 outputs the estimated psychological characteristic data to the administrator apparatus 180 during processing in S311 or after the processing in S311 is finished.

In place of a case where the answer data (one example of the subject intention data) and the estimated psychological characteristic data are outputted at different timings, integrated data of these data may be outputted. Further, the destination to which the answer data is outputted and the destination to which the estimated psychological characteristic data is outputted may be the same or different. Further, the estimated psychological characteristic data may be outputted to the subject apparatus 130 in place of or in addition to the administrator apparatus 180. With such processing, the subject 101 can get the estimated psychological characteristics of the subject 101 by answering the questions.

In the processing described with reference to FIG. 3, the measurement data from the subject apparatus 130 includes moving image data that expresses a moving image in which the subject 101 captured by the camera 102 is imaged. The behavioral feature generation unit 212 specifies related behavioral data for each one or a plurality of related behaviors based on the moving image data (recorded data). Various related behavioral data can be acquired from the moving image data and hence, it is expected that estimation accuracy of the psychological characteristics is enhanced.

Furthermore, in the processing described with reference to FIG. 3, the response control unit 220 may notify the subject 101 of a time limit for answering one or more questions. In general, in an interview or an investigation, an interviewer, a supervisor, or an instructor adjusts time while watching a situation of the subject 101, and leads the interview or the investigation such that the investigation of the personality trait is completed within a set time. However, in an automated interview or investigation, such a person does not exist (the other party of the subject 101 being a computer and not a human). Accordingly, it is practical to proceed the interview or the investigation in a state where a time limit is set to each question and each response. Such a difference in sensitivity to the time limit depending on the personality appears as behavioral features having different values related to the personality. As a result, it is expected that the estimation accuracy of the psychological characteristics is enhanced.

In the processing described with reference to FIG. 3, each of one or more behavioral features used for estimating the psychological characteristics of the subject 101 may be information where at least a part of the provided information is adjusted and/or a behavioral feature enhanced with an elapsed time with respect to such a behavioral feature. For example, the provision of the above-described guide contributes to enhancement of the behavioral feature. This is because the generation of a more accurate behavioral feature is expected with the provision of such a guide. The “enhancement” of the behavioral feature may be the relative increase of a behavioral feature and/or a weight thereof. Accordingly, the “enhancement” of the behavioral feature may include, for example, the relative decrease of one or more behavioral features other than the above-mentioned behavioral feature and/or a weight thereof.

The following example may be adopted as an example of enhancement of the behavioral feature. That is, for example, in S311, the psychological characteristic estimation unit 213 may enhance a behavioral feature corresponding to an elapsed time with respect to each of one or more behavioral features. The psychological characteristic estimation unit 213 may estimate the psychological characteristic of the subject 101 based on one or a plurality of behavioral features that include one or more enhanced behavioral features. In the interview or the investigation, the characteristic of the behavior of the subject 101 may change due to several reasons, for example, the reason that the subject 101 gets accustomed with to the atmosphere of the interview or the investigation (for example, the reason that the voice of the subject 101 becomes gradually smaller or larger, the reason that a respiration rate or a convergence speed in vital of the subject 101 changes, the reason that the gesture or the hand gesture of the subject 101 increases or decreases, the reason that the facial expression of the subject 101 changes, and the like.). Therefore, by using the behavioral feature that is enhanced corresponding to the elapsed time (for example, the degree of adaptation corresponding to the elapsed time) for estimation of the psychological characteristics (for example, by taking into account a change in the behavioral feature during the elapsed time), it is expected that the estimation accuracy of the psychological characteristics is enhanced. Further, the degree of appearance of the behavioral feature with respect to the time limit can also be reflected on the estimation of the psychological characteristics. Accordingly, it is expected that the estimation accuracy of the psychological characteristics is enhanced. In this paragraph, a part or all of the “one or a plurality of behavioral features” may be enhanced behavioral features. In other words, the “one or a plurality of behavioral features” may include a mixture of the enhanced behavioral features and the non-enhanced behavioral features.

As the estimation model stored in the estimation DB240, at least one of the following first to third estimation models may be stored in the estimation DB240. Each of the estimation models is a multiple regression equation model. However, the estimation models stored in the estimation DB240 may be other model in place of or in addition to at least one of the first to third estimation models.
First estimation model: y=∫(a(t)+b₁(t)x₁(t)+b₂(t)x₂(t)+b₃(t)x₃(t)+ . . . +bₙ(t)xₙ(t))dt
Second estimation model: y=a+b₁x₁+b₂x₂+b₃x₃+ . . . +bₙxₙ, and bₚxₚ=f(c_{p1[T1]}x_{p[T1]}, c_{p2[T2]}x_{p[T2]}, . . . , c_{pm[Tm]}x_{p[Tm]})
Third estimation model: y=a+b₁x₁+b₂x₂+b₃x₃+ . . . +bₙxₙ

The variables used in the above-mentioned estimation are as follows.
y is a psychological characteristic component (a dependent variable).
a is a constant.
bx is an explanatory variable.
x (for example, each of x₁, x₂, . . . , and xₙ) is a behavioral feature.
b (for example, each of b₁, b₂, . . . , and bₙ) and c (for example, each of cₚ₁, cₚ₂, . . . , and cₚₘ) are weighting coefficients respectively.
n and m are each an integer of 1 or more. Both n and m may vary depending on a psychological characteristic component. For example, n may differ depending on a psychological characteristic component, such as n=5 with respect to a certain psychological characteristic component, and n=3 with respect to another psychological characteristic component. The same goes for m.
t represents an elapsed time.
T (T1, T2, . . . , Tm) are time slots (examples of the elapsed time).
p is any desired integer selected from 1 to n.

Each of the first to third estimation models may be prepared for each psychological characteristic component. The same estimation model may be used for all of the plurality of psychological characteristic components (the values substituted for the explanatory variables may differ depending on the psychological characteristic component). Alternatively, one of the first to third estimation models may be used depending on a certain psychological characteristic component, and another one of the first to third estimation models may be used depending on another psychological characteristic component. As the plurality of psychological characteristic components constituting the psychological characteristic, components such as neurotic tendency, openness, sincerity, extraversion, and harmonicity may be adopted.

The psychological characteristic estimation unit 213 may estimate at least one psychological characteristic component using the first estimation model. The first estimation model includes a multiple regression model for each elapsed time. The multiple regression model for each elapsed time includes: n explanatory variables that correspond to n behavioral features on a 1:1 basis, (n being an integer of 1 or more, and being a value corresponding to the psychological characteristic component that corresponds to the multiple regression model); and weighting coefficients that are determined by the elapsed time with respect to the respective explanatory variables. According to the first estimation model, it is possible to estimate the psychological characteristics within the entire interview time frame while constantly changing the weighting coefficients with respect to the elapsed time. That is, it is possible to expect the highly accurate psychological characteristic estimation. For example, the first elapsed time may be a first time range from the start of the interview to a first point of time for a certain behavioral feature, and the second elapsed time may be a second time range from k minutes after the start of the interview to a second point of time for another behavioral feature. The time between points of time in the first elapsed time and the time between points of time in the second elapsed time may be continuous, may be discrete from each other, or may overlap with each other. For each elapsed time, the start and the end of the elapsed time may be dynamically determined based on the behavioral feature or the like. Further, the elapsed time is typically dynamic and hence, the length of the elapsed time and the number of elapsed times may differ between a case where the first estimation model is used for estimating a certain psychological characteristic component and a case where the first estimation model is used for estimating another psychological characteristic component.

The psychological characteristic estimation unit 213 may estimate at least one psychological characteristic component using the second estimation model. The second estimation model includes n multiple regression models that correspond to n behavioral features on a 1:1 basis (n being an integer of 1 or more). With respect to each of the n multiple regression models, the multiple regression model includes: m weighting coefficients that correspond to m time slots (m being an integer of 2 or more) constituting time (for example, a time for evaluating the subject such as an interview time) on a 1:1 basis; and explanatory variables that are behavioral features that correspond to the multiple regression models with respect to the respective m weighting coefficients. With such processing, psychological characteristic estimation that takes into account the elapsed time can be performed with a small calculation resource (with a small arithmetic operation load for the arithmetic operation apparatus 115) compared to the first estimation model. For example, the lengths of m time slots may be the same or differ from each other with respect to one psychological characteristic component. In addition, the lengths of the m time slots may be substantially equal with respect to all psychological characteristic components, or may differ from each other depending on the psychological characteristic components. In the latter case, it is desirable that the lengths of the respective time slot are appropriate lengths depending on the behavioral features that are the explained variables. Further, in the estimation using the second estimation model with respect to a certain psychological characteristic component, a certain explanatory variable may be expressed by a formula: bₚxₚ=c_{p1[1]}x_{p[1]}+c_{p2[T2]}x_{p[T2]}+ . . . +c_{pm[Tm]}x_{p[Tm]}, and another explanatory variable may be expressed by a different formula.

With respect to the estimation of at least one psychological characteristic component where it is preferable to take into account the elapsed time, the psychological characteristic estimation unit 213 may select which one of the first estimation model and the second estimation model is to be use based on a model selection policy (for example, which one the priority is to be assigned between the calculation load and the estimation accuracy) that includes a relationship (typically, a magnitude relationship) between an arithmetic operation load (for example, a processor usage rate) of the arithmetic operation apparatus 115 and a threshold of the arithmetic operation load. For example, in a case where an arithmetic operation load of the arithmetic operation apparatus 115 is less than a threshold, the psychological characteristic estimation unit 213 may select a first estimation model that is expected to exhibit higher estimation accuracy although the first estimation model requires more calculation resources, and may estimate a psychological characteristic component using the first estimation model. On the other hand, in a case where the arithmetic operation load of the arithmetic operation apparatus 115 is equal to or more than the threshold, the psychological characteristic estimation unit 213 may select the second estimation model that requires the less calculation resource, and may estimate the psychological characteristic component using the second estimation model. By performing the above-mentioned processing, an optimal estimation model can be selected corresponding to the arithmetic operation situation and hence, it is expected that the estimation that corresponds to the arithmetic operation situation can be performed.

In the processing described hereinafter, at least one of the following technical features may be adopted.
The behavioral feature generated based on the moving image data may be at least one of an eye motion, an eyebrow motion, a mouth motion, a nose motion, and a change in complexion.
The behavioral feature generated based on a moving image data may be at least one of a change in pupil, a change in line of sight, a degree of gazing, and involuntary eye movement during fixation.
The behavioral feature generated based on a moving image data may be at least one of a head motion, a body motion, a shoulder motion, an arm motion, a hand motion, and a position of the subject 101 with respect to an angle of view of the camera 102.
The behavioral feature generated based on the moving image data may be at least one of a pulse rate, a degree of stress, and a respiratory rate estimated from a change in complexion between video frames in the moving image data.

The personality trait estimation system 100 may be a server system, and a plurality of client systems may be systems of a plurality of organizations (for example, a plurality of companies) having different purposes, different questions, and the like in interviewing (interview). The estimation of the personality trait is performed using a behavioral feature that is a “nonverbal features” based on data of “related behaviors” that do not depend on questions and answers. Accordingly, the psychological characteristics of the subject can be estimated without making the subject perform a behavior dedicated for estimating the psychological characteristics.

In the present embodiment, the personality trait estimation is performed using a machine learning model such as the above-described estimation model. The machine learning model is a model that does not depend on questions and answers, more specifically, a model based on a nonverbal features based on related behavioral data. Accordingly, although the contents of questions and the like in an interview differ between the respective organizations in general, according to the present embodiment, it is not necessary to prepare different (learn) machine learning models for the respective organizations. Machine learning models of different types such as a neural network may be also used in place of the regression equation model described above.

Furthermore, in a self-interview where a virtual robot such as an avatar serves as an interviewer and the subject 101 is a person who receives an interview, the behavior (reaction) of the subject 101 tends to be small unlike an interview where a human serves as an interviewer. Accordingly, although there is a possibility that it is difficult to accurately estimate the psychological characteristics of the subject 101 from the moving image data (recorded data) of the interview, in this embodiment, such a problem also can be overcome.

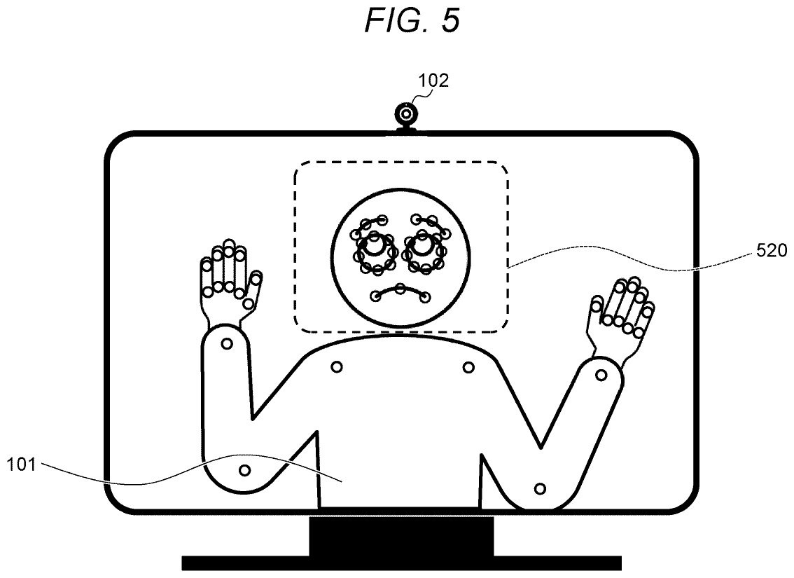

That is, as illustrated in FIG. 5, the behavioral feature generation unit 212 sets an enhancement amount of the behavioral feature based on related behavioral data in a head region 520 of the subject 101 imaged in the motion image (recorded image) larger than an enhancement amount of the behavioral feature based on the related behavioral data other than the head region 520 of the subject 101. The “enhancement amount” is an additional amount of the behavioral feature or its weight. The enhancement amount may be zero, a positive value or a negative value. For example, the behavioral feature generation unit 212 may set the weight of the behavioral feature based on the related behavioral data in the head region 520 of the subject 101 larger than the weight of the behavior feature mount based on the related behavioral data in regions other than the head region 520 of the subject 101. The psychological characteristic estimation unit 213 performs the personality trait estimation using the estimation model based on the behavioral feature and its weight relating to the head region 520 and the behavioral feature and its weight relating to regions other than the head region 520. In each frame of the moving image data, the “head region” may be a region that includes the entire head of the subject 101 or a region that is detected as the head.

Specifically, for example, as illustrated in FIG. 5, the behavioral data generation unit 290 receives the measurement data including the moving image data of the subject 101 during the interview, and recognizes respective feature points such as the entire head, eyes, a nose, a mouth, eyebrows, shoulders, hands, and fingers for each frame (image) in the moving image data, and generates the positions and sizes of the recognized feature points and/or portions as the related behavioral data. In recognizing which part of the image corresponds to which portion of the subject 101, an existing technique may be used. For example, a technique may be used where landmarks in regions such as a head, a face, hands and the like are recognized by image recognition processing. In this manner, the related behavioral data may be generated with respect to the entire head, the eyes, the nose and the like respectively. The related behavior values such as a total motion amount, a motion speed, a motion acceleration, an amount of change in size, a total rotational amount, a rotational speed, a rotational acceleration and the like may be acquired for each of M frames (M being a natural number) in the moving image data. Accordingly, the time series of the related behavior value can be obtained for each kind of the related behavior value. The behavioral feature generation unit 212, with respect to at least one type of related behavior value such as a total motion amount, a motion speed, a motion acceleration, an amount of change in size, a total rotational amount, a rotational speed, a rotational acceleration and the like, acquires at least one of the number of times of detection, an average value, a standard deviation, a minimum value, a first quartile, a second quartile, a median value, a third quartile, a maximum value and an initial value, and the like as behavioral features based on time series of related behavior values. It is unnecessary to limit the acquired behavioral feature to one behavioral feature, and it is possible to estimate psychological characteristic by integrating versatile behavioral features.

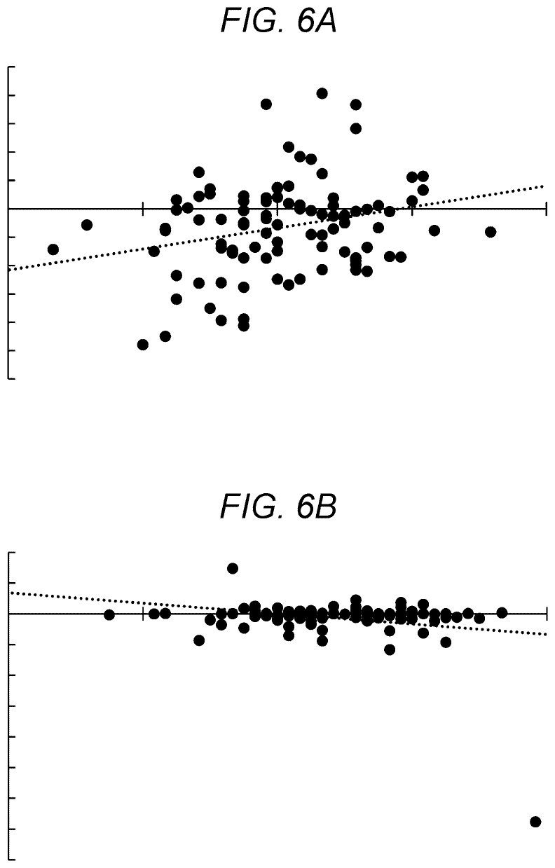

The reason that an enhancement amount of the behavioral feature relating to the head region 520 is set larger than an enhancement amount of the behavioral feature relating to regions other than the head region 520 is as follows. That is, as illustrated in FIG. 6A, the distribution of the behavioral feature obtained from the motions (for example, movements of the entire head, eyes, nose or mouth) in the head region has normality with respect to the psychological characteristic component. In the graph, a psychological characteristic component is taken on an axis of abscissas and a behavioral feature is taken on an axis of coordinates. Specifically, the tendency is obtained where the higher a numerical value of the psychological characteristic component, the larger the behavioral feature becomes. On the other hand, as illustrated in FIG. 6B, with respect to the distribution of the behavioral feature obtained from the movement of the portion (for example, the shoulder) other than the head region, no normality is observed related to the psychological characteristic component. In the graph, a psychological characteristic component is taken on an axis of abscissas and a behavioral feature is taken on an axis of coordinates. Further, in a case where a portion other than the head region moves relatively largely, the movement is likely to be so-called noise, and hence, the estimation accuracy of the personality trait may be lowered. Accordingly, in a state where there is a tendency that the movement of the subject 101 is small as a whole, setting an enhancement amount of the behavioral feature obtained from the movement in the head region larger than an enhancement amount of the behavioral feature obtained from the movement of regions other than the head region has the technical significance.

The behavioral feature generation unit 212 sets an enhancement amount of the behavioral feature indicating how large the behavior of the subject 101 is than an enhancement amount of the behavioral feature that is how small the behavior of the subject 101 is. The reason is as follows. In a use case (for example, a self-interview) where tendency is observed that the behavior of the subject 101 is small, noises are easily generated as a related behavior value and hence, it is difficult to acquire a significant behavioral feature. However, the behavioral feature from a viewpoint that how large the behavior technical feature is taken can easily extract the significant behavioral feature with small noises. In this manner, by increasing an enhancement amount of the behavioral feature that indicates how large the behavior of the subject 101 becomes, the enhancement of estimation accuracy of the psychological characteristic can be expected. The behavioral feature where how large the behavior of the subject is large is at least one of a time series average value, a standard deviation, a median value, a third quartile and a maximum value of the related behavior value, (for example, a total operation amount or an operational speed or the like). The behavioral feature indicating how small the behavior of the subject is at least one of the minimum value, the first quartile, and the second quartile of the time series of the related behavior values.

The psychological characteristic estimation unit 213 may acquire the value of the psychological characteristic component that is the explained variable by inputting the weighted behavioral feature to the estimation model. An estimation model may be prepared for each psychological characteristic component. The same estimation model may be used for all of the plurality of psychological characteristic components (the value substituted for the explanatory variable may differ depending on the psychological characteristic component).

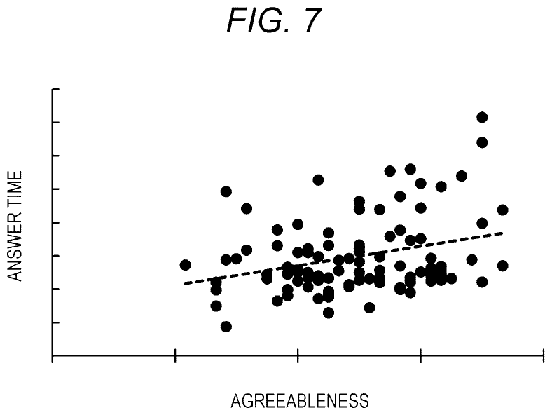

As a nonverbal features (behavioral feature) based on related behavioral data that does not depend on questions and answers, as illustrated in FIG. 4, a feature value regarding an answer time may be adopted as the behavioral feature in place of or in addition to the behavioral feature of active behavior such as movement of the subject 101. Specifically, the related behavioral data may be data indicating a length of an answer time from a point of time that a question is provided (for example, displayed) to the subject 101 to a point of time that an answer to the question is given. The behavioral feature may include a behavioral feature based on an answer time or an amount of change in the answer time. As such a behavioral feature, it is possible to adopt an average value, a standard deviation or the like with respect to an answer time from a point of time that a question is displayed to a point of time that an answer is given, or an amount of change in the answer time. According to an example illustrated in FIG. 7, the longer the answer time, the higher the harmonicity tends to become. Accordingly, it is possible to estimate at least the level of harmonicity among the psychological characteristic components using the estimation model based on the behavioral feature such as the average value or the standard deviation of the answer time. Furthermore, the answer time may be measured as, for example, a time from a point of time that a question is displayed to a point of time that a completion button of the answer to the question is pressed. The measurement (acquisition) of the answer time is more accurate and exhibits a lower load than the recognition of landmarks such as a face and a body from recorded data. Furthermore, as described above, a predetermined restriction related to time such as a time limit of answering to a question may be given to the subject 101, or the behavioral feature related to the answer time may be extracted based on the answer time and the restriction imposed on the time.

### Second Embodiment

A second embodiment will be described. In the description of the second embodiment, the description is made mainly focusing on differences between the first embodiment and the second embodiments, and the description is omitted with respect to the substantially same configuration.

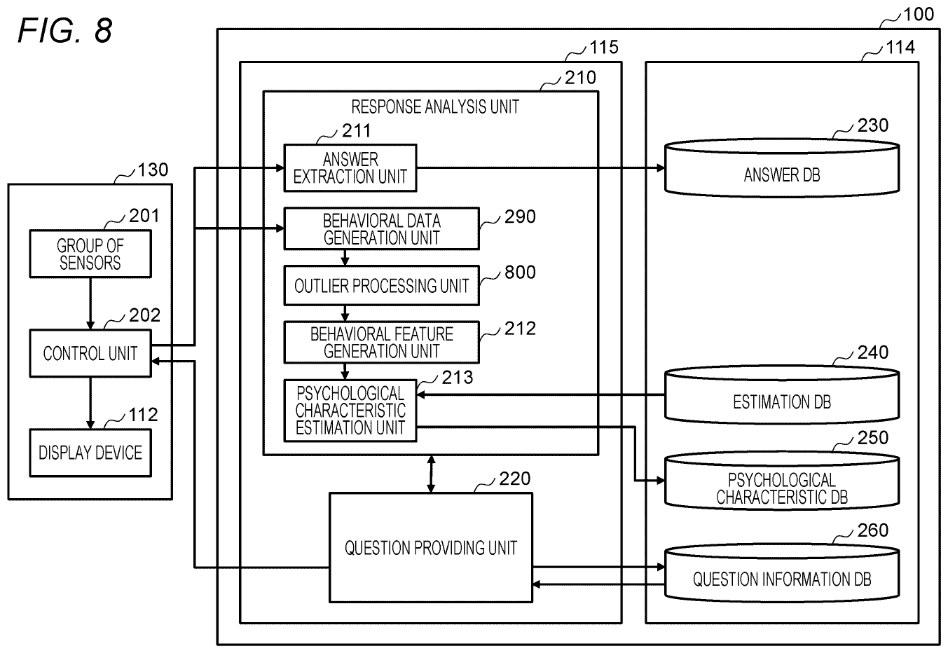

FIG. 8 illustrates data and functions in the entire system according to the second embodiment.

In the self-interview, there is a tendency that the behavior of the subject 101 is small as described above. Accordingly, an outlier (noise) in a time series example of a related behavior value adversely affects the accuracy of a behavioral feature. Examples of the cause of the outlier include: failure of recognition of a facial landmark (target point) due to disturbance, video disturbance due to system noise during communication or the like, or a connecting portion between moving images in a case where a moving image for each question is joined to one moving image.

To cope with such a case, the response analysis unit 210 includes an outlier processing unit 800. The outlier processing unit 800 processes an outlier of a related behavior value that is obtained from the related behavioral data generated by the behavioral data generation unit 290. Since the behavioral feature is extracted from the plurality of related behavior values in which the outliers have been processed, it is possible to expect the enhancement in accuracy of the behavioral feature.

Outliers caused by the above-mentioned example occur in consecutive Y frames (Y being an integer greater than or equal to 2 (for example, 3 to 5)). Accordingly, the outlier processing unit 800 determines whether there is a sudden change (signal change) in the related behavior value in consecutive Y frames. When a result of the determination is true, the outlier processing unit 800 corrects outliers in the several frames.

Specifically, for example, the outlier processing unit 800 determines, for each measurement window range 903 of the moving image data, whether a change in the related behavior value of the consecutive Y frames constituting the measurement window range satisfies a condition. In a case where a result of the determination is true, the outlier processing unit 800 detects the related behavior value satisfying such a condition as an outlier and corrects the detected outlier. To be more specific, for example, outlier value processing unit 800, with respect to the measurement window range 903 (for example, a time range of several consecutive frames), estimates a standard deviation of each sample (related behavior value) with respect to the median value within a measurement window range 903. Then, the outlier processing unit 800 determines whether or not a sample that is over N times larger than a standard deviation from the median value exists within the measurement window range 903. A sample whose determination result is true is a sample (related behavior value) as an outlier. In a case there is an outlier, the outlier processing unit 800 corrects the outlier. For example, the outlier processing unit 800 replaces the outlier with a median value. Y may be an arbitrary integer value, for example, +1 to 3. Accordingly, the measurement window range 903 may be a range of +1 to 3 frames. N may be a predetermined value larger than 1. For example, N may be 10 (N=10).

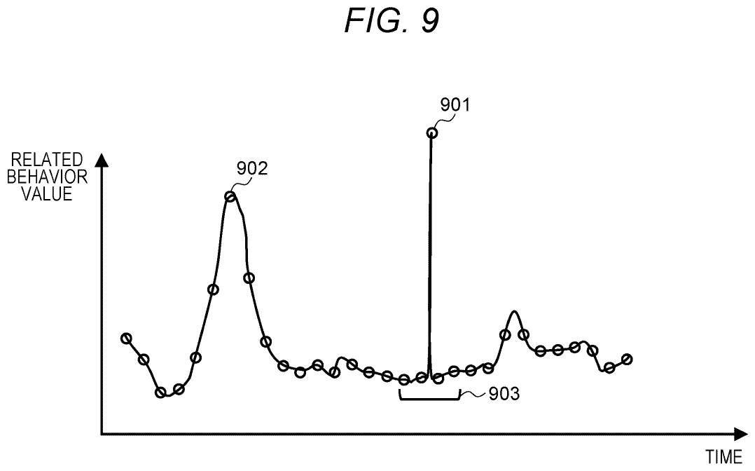

In this manner, the presence or absence of an outlier is determined depending on whether or not a sudden change in signal occurs in the measurement window range 903. As a result, according to the example illustrated in FIG. 9, the sample 901 after a steep change is an outlier. The sample 902 which does not correspond to such a change although a value is comparatively large is not an outlier.

In a case where a characteristic pattern is generated on the image in the noise frame, the outlier processing unit 800 may identify a frame that becomes an outlier value by image recognition, and may correct a related behavior value obtained from the frame.

Although several embodiments have been described heretofore, these embodiments are provided for exemplifying purpose, and it is not intended to limit the scope of the present invention only to these embodiments. The present invention can be carried out in various other modes.

For example, in all embodiments, behavioral features are generated from related behavioral data of the subject 101 that change within a limited time of an interview or the like, and psychological characteristics are estimated based on the generated behavioral features. Accordingly, the objective and high accurate psychological characteristic estimation can be automatically limited within a limited time.

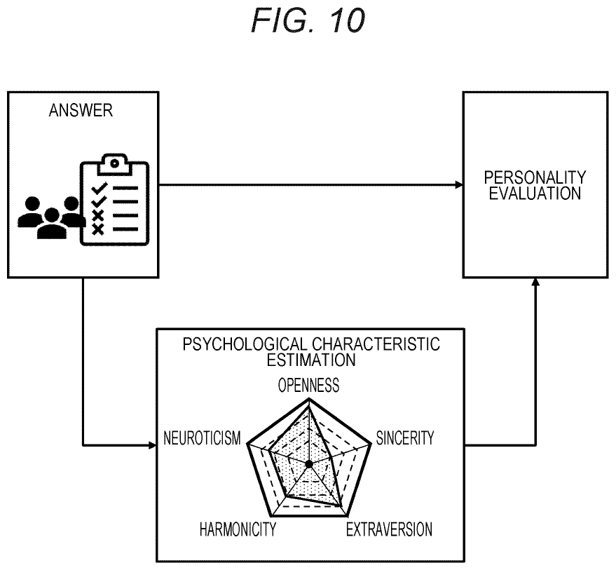

Further, at least one of the first and second embodiments, a practical application illustrated in FIG. 10 may be adopted. That is, a personality evaluation system may exist. The personality evaluation system performs personal evaluation (for example, decision as to whether or not the subject is employed) based on answer data of the subject and the estimated psychological characteristic data of the subject. The personality evaluation system may be a function within the personality trait estimation system 100, or may be a function outside the personality trait estimation system 100 (for example, a physical computer system, or a logical computer system such as a cloud computing service). The personality trait estimation system 100 may, for each subject, extract related behavioral data from measurement data for each answer to the question, and may output the answer data expressing the answers to the respective questions and an estimated psychological characteristic data expressing psychological characteristic estimated based on the related behavioral data for each answer. By performing such a practical application, the possibility of realizing automatic personality evaluation system of each subjects can be enhanced by a technical means referred to as the personality trait estimation system 100.

What is claimed is:

1. A personality trait estimation system comprising:
an interface apparatus that is connected to a subject apparatus formed of an apparatus that includes one or a plurality of sensors; and
an arithmetic operation apparatus configured to be connected to the interface apparatus, wherein
the arithmetic operation apparatus is configured to receive measurement data related to a behavior performed by a subject and based on measurement performed by one or a plurality of sensors from the subject apparatus via the interface apparatus,
the arithmetic operation apparatus is configured to generate related behavioral data of a related behavior that is an entire or a partial behavior excluding instruction of a subject intention from the measurement data,
the arithmetic operation apparatus is configured to acquire one or a plurality of behavioral features that are respectively nonverbal features based on related behavioral data with respect to one or each of a plurality of related behaviors, and is configured to estimate a psychological characteristic of the subject based on the one or the plurality of behavioral features, and
the arithmetic operation apparatus is configured to output estimated psychological characteristic data that is data expressing the psychological characteristic that is estimated.

2. The personality trait estimation system according to claim 1, wherein
the one or the each of plurality of sensors includes a camera:
the measurement data includes moving image data that is data expressing a moving image in which the subject imaged by the camera appears:
the arithmetic operation apparatus is configured to recognize a region of a head itself of the subject or a region that includes the head of the subject from the moving image data,
the arithmetic operation apparatus is configured to set an enhancement amount of a behavioral feature calculated with respect to the head region larger than an enhancement amount of a behavioral feature calculated related to a region other than the head region.

3. The personality trait estimation system according to claim 1, wherein
the arithmetic operation apparatus is configured to set an enhancement amount of the behavioral feature indicating how large the behavior of the subject is larger than an enhancement amount of the behavioral feature indicating how small the behavior of the subject is.

4. The personality trait estimation system according to claim 3, wherein
a related behavior value that is a value of a related behavior is acquired from related behavioral data of the related behavior,
a behavioral feature indicating how large the behavior of the subject is at least one of an average value, a standard deviation, a median value, a third quartile and a maximum value of a time series of the related behavior value, and a behavioral feature indicating how small the behavior of the subject is at least one of a minimum value, a first quartile, and a second quartile of a time series of related behavior value.

5. The personality trait estimation system according to claim 1, wherein
the related behavioral data is data indicating a length of an answer time from a point of time that a question is provided to the subject to a point of time that an answer to the question is given, and
the one or the plurality of behavioral feature include a behavioral feature based on the answer time or an amount of change in the answer time.

6. The personality trait estimation system according to claim 1, wherein
the one or more sensors include a camera,
the measurement data includes moving image data that is data expressing a moving image in which the subject imaged by the camera appears,
a related behavior value that is a value of a related behavior is acquired from related behavioral data,
the behavioral feature is acquired from a time series of related behavior value, and
the arithmetic operation apparatus is configured to determine, for each measurement window range of the moving image data, whether a change in a related behavior value of consecutive frames constituting the measurement window range satisfies a condition, is configured to detect a related behavior value satisfying the condition as an outlier when a result of the determination is true, and is configured to correct the detected outlier.

7. The personality trait estimation system according to claim 6, wherein
the arithmetic operation apparatus is configured to estimate, for each of the measurement window ranges, a standard deviation of each related behavior value with respect to a median value within the measurement window range, is configured to determine whether or not the related behavior value that is over N times larger than the standard deviation from the median value is within the measurement window range (N being a predetermined value larger than 1), and in a case where the determination result is an outlier that is a true related behavior value, the arithmetic operation apparatus is configured to replace the outlier with the median value.

8. The personality trait estimation system according to claim 1, wherein
the measurement data is data relating to a behavior performed by the subject in a self-interview that is an interview where a virtual robot is an interviewer and the subject is a person who receives an interview and measured by the one or the plurality of sensors.

9. A personality trait estimation method performed by a computer, the personality trait estimation method comprising:
receiving measurement data related to a behavior performed by a subject and based on measurement performed by one or a plurality of sensors from a subject apparatus;
generating related behavioral data of related behaviors that is all or part of behaviors excluding designation of an intension of the subject from the measurement data,
acquiring one or a plurality of behavioral features that are respectively nonverbal features based on related behavioral data with respect to one or the plurality of respective related behaviors, estimating psychological characteristic of the subject based on the one or the plurality of behavioral features, and outputting estimated psychological characteristic data that expresses the estimated psychological characteristic.

10. A computer program that allows a computer to perform:
receiving measurement data related to a behavior performed by a subject and based on measurement performed by one or a plurality of sensors from a subject apparatus;
generating related behavioral data of related behaviors that is all or part of behaviors excluding designation of an intension of the subject from the measurement data,
acquiring one or a plurality of behavioral features that are respectively nonverbal features based on related behavioral data with respect to one or the plurality of respective related behaviors, and estimating psychological characteristic of the subject based on the one or the plurality of behavioral features, and outputting estimated psychological characteristic data that expresses the estimated psychological characteristic.
